(19) United States
(12) Patent Application Publication
(10) Pub. No.: US 2019/0046195 A1
(43) Pub. Date: Feb. 14, 2019

(54) DEVICES AND METHODS FOR INHIBITING SCAR FORMATION IN A HEALING WOUND OR INCISION

(71) Applicant: ZipLine Medical, Inc., Campbell, CA (US)

(72) Inventors: Amir BELSON, Savyon (IL); Keiichiro ICHIRYU, Campbell, CA (US)

(21) Appl. No.: 16/161,598

(22) Filed: Oct. 16, 2018

Related U.S. Application Data
(63) Continuation of application No. PCT/US17/28537, filed on Apr. 20, 2017.
(60) Provisional application No. 62/361,981, filed on Jul. 13, 2016.
(60) Provisional application No. 62/325,902, filed on Apr. 21, 2016.

(51) Int. Cl. A61B 17/08

(52) U.S. Cl. CPC A61B 17/085; A61B 2017/00862; A61B 2017/086

(57) ABSTRACT

Devices and methods of closing a wound or incision and inhibiting scar formation in the closed wound or incision are provided. First and second base panels of a device are adhered to skin on first and second lateral sides of the wound or incision, respectively. The first and second panels are coupled together with one or more elastic lateral ties or straps positioned across the wound or incision. A lateral compressive force is applied on the wound or incision with the one or more lateral ties. The lateral compressive force is maintained for a period of time as the wound or incision heals. The maintained lateral compressive force inhibits scar formation in the healing wound or incision.

### CROSS-REFERENCE

This application is a continuation of PCT Application No. PCT/US17/28537, filed Apr. 20, 2017, which claims the benefit of U.S. Provisional Application Nos. 62/325,902, filed Apr. 21, 2016, and 62/361,981, filed Jul. 13, 2016, the contents of which are incorporated herein by reference.

The subject matter of this application is related to the subject matter of the following U.S. patents and co-pending U.S. patent applications: application Ser. No. 13/685,909, filed Nov. 27, 2012 [Attorney Docket No. 35383-705.301]; application Ser. No. 13/414,176, filed 7 Mar. 2012 and issued as U.S. Pat. No. 9,179,914 on 10 Nov. 2015 [Attorney Docket No. 35383-706.301]; application Ser. No. 14/958,803, filed 3 Dec. 2015 [Attorney Docket No. 35383-706.302]; application Ser. No. 14/958,818, filed 3 Dec. 2015 [Attorney Docket No. 35383-706.303]; application Ser. No. 14/851,059, filed 11 Sep. 2015 [Attorney Docket No. 35383-706.501]; application Ser. No. 13/286,757, filed 1 Nov. 2011 and issued as U.S. Pat. No. 8,323,313 on 4 Dec. 2012 [Attorney Docket No. 35383-709.201]; application Ser. No. 14/625,366, filed 18 Feb. 2015 [Attorney Docket No. 35383-709.301]; application Ser. No. 13/665,160, filed 31 Oct. 2012 [Attorney Docket No. 35383-709.501]; application Ser. No. 14/180,564, filed 14 Feb. 2014 and issued as U.S. Pat. No. 9,089,328 on 28 Jul. 2015 [Attorney Docket No. 35383-709.502]; and application Ser. No. 14/180,524 filed 14 Feb. 2014 and issued as U.S. Pat. No. 9,050,086 on 9 Jun. 2015 [Attorney Docket No. 35383-712.201] which are incorporated herein by reference.

The subject matter of this application is related to the subject matter of the following PCT applications: application Serial No. PCT/US2011/40213, filed Jun. 13, 2011 [Attorney Docket No. 35383-705.601]; application Serial No. PCT/US2010/000430, filed 3 May 2010 [Attorney Docket No. 35383-706.601]; application Serial No. PCT/US2015/049671, filed 11 Sep. 2015 [Attorney Docket No. 35383-706.602]; application Serial No. PCT/US2012/062820, filed 31 Oct. 2013 [Attorney Docket No. 35383-709.601]; application Serial No. PCT/US2013/067563, filed 30 Oct. 2013 [Attorney Docket No. 35383-709.602]; application Serial No. PCT/US2014/016587, filed 14 Feb. 2014 [Attorney Docket No. 35383-712.601]; application Serial No. PCT/US2015/010188, filed 5 Jan. 2015 [Attorney Docket No. 35383-713.601]; and application Serial No. PCT/US2015/028066, filed 28 Apr. 2015 [Attorney Docket No. 35383-714.601] which are incorporated herein by reference.

### BACKGROUND

### 1. Field of the Invention

The present disclosure relates to medical devices, systems, and methods for wound closure and facilitating healing. In particular, devices, systems, and methods for inhibiting scar formation in a healing wound or incision are disclosed.

Scarring is a natural response to the healing of wounds in a patient's skin. The wound healing process can be divided into three successive stages. A first inflammatory stage begins immediately after the injury which caused the wound. The inflammatory stage lasts up to a week during which time cellular processes remove damaged tissue and foreign matter from the wound. The inflammatory stage is followed by a proliferative stage which is characterized by fibroblast proliferation and the production of collagen and proteoglycans. The proliferative stage can last from days to weeks and is typically the stage during which hypertrophic scar formation begins, typically as a result of excess production of the extra cellular matrix which is synthesized to heal the wound. Such scars are referred to “hypertrophic scars.” After the proliferative stage, a remodeling phase begins where the matrix which was produced in the proliferative is remodeled into an organized, cross-linked structure which increases the mechanical strength of the healed tissue.

Scars which are formed during the healing response are often treated after the healing is complete by cosmetics or surgical treatment. Cosmetic treatments are at best temporary solutions, and the need to have subsequent surgery is both inconvenient and a risk to the patient. It would therefore be desirable to provide methods and apparatus which could diminish scar formation during the healing process itself so that such subsequent procedures would be unnecessary or at least reduced in scope.

### 2. Description of the Background Art

WO/2011/019859 describes a device for inhibiting scarring in skin wounds, where the device is fixed to the skin and applies an outward force to spaced-apart skin locations. U.S. Pub. No. 2008/0069855 describes materials that will prevent scars and tissue adhesions. U.S. Pat. Nos. 7,511,185, 4,702,251, 4,539,990 and 4,535,772 describe wound healing devices that apply laterally inwardly opposed forces to the tissue on either side of the wound. Further references of interest include: U.S. Patent Publications Nos. 2008/0033334, 2008/0014396, 2008/0147115, 2008/0228219, 2009/0099496, 2009/0299257, 2009/0299303, 2010/0121286, and US2009/0299255; U.S. Pat. Nos. 4,605,005, 4,815,468, 5,514,155, 5,665,108, 6,007,564, and 6,176,868; and PCT Publications Nos. WO 96/029013 and WO 2008/060532.

### SUMMARY

The present disclosure provides devices, systems, and methods for inhibiting scar formation during the healing of a closed wound or surgical incision. The term “scar” as intended includes not only common hypertrophic scars which occur from cuts, abrasion, and the like, but also to include keloids which are hyperplastic masses that occur in the dermis and adjacent subcutaneous tissue in certain individuals, typically following trauma.

The devices, systems, and methods of the present disclosure treat and inhibit scar formation in healing wounds or incisions by applying a laterally compressive force across the healing wound or incision in order to relieve tension in the tissue across or surrounding the wound or incision, usually during the proliferative and/or remodeling stages of the healing process. Treatment during the late portions of the inflammatory stage might also be useful, and in some instances treatment after remodeling might also be performed. For example, lateral compression may be applied to the wound at or near the end of the inflammatory stage, typically at a time in the range from 1 day to 30 days after the wound has occurred or the incision made and first began to heal. The lateral compression will typically be applied after the same incision or wound closure device has been closed using sutures, staples, or closure devices have been used to close the incision or wound, often after the sutures, staples, or closure devices have been removed. After commencing treatment, the lateral compression may be continued to be applied for a period of time in the range from 1 day to 21 days, or longer, and the forces may be increased, decreased, or held constant during the treatment period. Alternatively or in combination, the devices, systems, and methods may off-load tension from the incision site to facilitate wound healing and reduce scar formation.

Aspects of the present disclosure provide methods of closing a wound or incision and inhibiting scar formation in the closed wound or incision. First and second base panels adhered to skin on first and second lateral sides of the wound or incision, respectively, may be provided. The first and second panels may be coupled together with one or more elastic lateral ties or straps positioned across the wound or incision. A lateral compressive force may be applied on the wound or incision with the one or more lateral ties or straps. The lateral compressive force may be maintained for a period of time as the wound or incision heals, such as from 1 day to 21 days.

The one or more lateral ties or straps may comprise a plurality of axially-adjacent lateral ties or straps. In some embodiments, wherein the plurality of axially-adjacent lateral ties or straps are provided by an elastic film having a plurality of cutouts thereon such that the elastic film has a serpentine pattern and comprises a plurality of discrete side regions and a plurality of lateral straps distributed between the side regions. The one or more elastic lateral ties or straps may elastically apply the compressive force through the first and second base panels adhered to the skin. The first and second base panels may be stretched apart laterally to place the one or more lateral ties in tension prior to being adhered to the first and second lateral sides of the wound or incision.

The first and second base panels may each comprise force distribution structures coupling the one or more lateral ties or straps to the first and second base panels. The force distribution structures may be axially distributed over the lengths of the first and second base panels. One or more of the first or second base panels may be axially stretched, and the one or more second base panels may have preferentially stretching regions between axially adjacent force distribution structures, such as one or more perforations or other weakened regions through the base panel body.

One or more of sutures, staples, or a closure device may be removed from the skin prior to the first and second base panels being adhered to the skin. The wound or incision adjacent the applied base panels may be present on a joint of a patient such as a knee. Applying the lateral compressive force on the wound or incision with the one or more lateral ties may cause the wound or incision to pucker. One or more of the base panels may be axially stretched.

Aspects of the present disclosure may also provide methods of closing a wound or incision and inhibiting scar formation in the closed wound or incision. In an exemplary method, a first base panel of a scar inhibition device may be adhered to skin on a first lateral side of the wound or incision. The first base panel and a second base panel of the scar inhibition device may be stretched apart to place one or more elastic lateral ties or straps coupling the first and second base panels together in tension. The scar inhibition device may be positioned such that the one or more lateral ties or straps are positioned across the wound or incision. The second base panel may be adhered to skin on a second lateral side of the wound or incision opposite the first lateral side, thereby having the one or more lateral ties or straps apply a lateral compressive force on the wound or incision. The lateral compressive force may be maintained for a period of time as the wound or incision heals, such as from 1 day to 21 days.

The first base panel may be adhered to the first lateral side of the wound or incision before the second base panel is adhered to the second lateral side of the wound or incision. The first and second base panels may be stretched laterally apart after the first base panel is adhered to the first lateral side of the wound or incision. The first base panel may be adhered to the first lateral side of the wound or incision concurrently with the second base panel being adhered to the second lateral side of the wound or incision. The first and second base panels may be stretched apart before the first and second base panels are adhered to the skin.

The one or more lateral ties or straps may comprise a plurality of axially-adjacent lateral ties. The one or more elastic lateral ties or straps may elastically apply the compressive force through the first and second base panels adhered to the skin. The first and second base panels may each comprise force distribution structures coupling the one or more lateral ties to the first and second base panels. The force distribution structures may be axially distributed over the lengths of the first and second base panels. One or more of the first or second base panels may be axially stretched, and the one or more second base panels may have preferentially stretching regions between axially adjacent force distribution structures, such as one or more perforations or other weakened regions through the base panel body.

The method may further comprise a step of removing one or more of staples, sutures, or a closure device from the wound or incision or skin adjacent thereto prior to adhering the first and second base panels to the skin. The wound or incision adjacent the applied base panels may be present on a joint of a patient such as a knee. Applying the lateral compressive force on the wound or incision with the one or more lateral ties may cause the wound or incision to pucker. One or more of the base panels may be axially stretched.

Aspects of the present disclosure may also provide methods of closing a wound or incision and inhibiting scar formation in the closed wound or incision. In an exemplary method, a first base panel of a scar inhibition device may be adhered to skin on a first lateral side of the wound or incision, and a second base panel of the scar inhibition device may be adhered to skin on a second lateral side of the wound or incision. An elastic panel coupled to the first base panel may be stretched to place the elastic panel in tension. The tensioned elastic panel may then be coupled to the second base panel, thereby applying a lateral compressive force on the wound or incision through the first and second base panels coupled to one another by the tensioned elastic panel. The lateral compressive force may be maintained for a period of time as the wound or incision heals, such as from 1 day to 21 days.

The first base panel may be adhered to the first lateral side of the wound or incision before the second base panel is adhered to the second lateral side of the wound or incision. Or, the first base panel may be adhered to the first lateral side of the wound or incision concurrently with the second base panel being adhered to the second lateral side of the wound or incision.

The elastic panel may comprise an elastic film having a plurality of cut-outs such that the elastic film has a serpentine pattern having a plurality of lateral straps and a plurality of side regions, the side regions being configured to be adhered to the first or second base panel. The elastic panel may apply the compressive force through the first and second base panels adhered to the skin.

One or more of the first or second base panels may be axially stretched.

In some embodiments, one or more of staples, sutures, or a closure device are removed from the wound or incision or skin adjacent thereto prior to adhering the first and second base panels to the skin. The wound or incision adjacent the applied base panels may be present on a joint of a patient such as a knee. Applying the lateral compressive force on the wound or incision with the one or more lateral ties may cause the wound or incision to pucker. One or more of the base panels may be axially stretched.

To couple the elastic panel to the second base panel, a release liner may be removed from an adherent bottom surface of the elastic panel and the adherent bottom surface of the elastic panel may be adhered to the second base panel. To stretch the elastic panel, a pull tab coupled to the elastic panel may be pulled laterally. The pull tab may be removed from the elastic panel after the elastic panel is coupled to the second base panel.

Aspects of the present disclosure may also provide devices for closing a wound or incision and inhibiting scar formation in the closed wound or incision. An exemplary closure device may comprise first and second base panels and one or more elastic ties coupling couple the first and second base panels to one another. The first and second base panels may be configured to be adhered to skin on first and second lateral sides of the wound or incision, respectively. Each base panel may comprise an upper surface and an adherent lower surface. When the one or more elastic lateral ties are stretched and placed into tension, the one or more elastic lateral ties may apply a lateral compressive force on the wound or incision through the first and second base panels adhered to the skin. The plurality of elastic lateral ties may comprise a plurality of axially-adjacent elastic lateral ties. The first and second base panels may each comprise force distribution structures coupling the one or more lateral ties to the first and second base panels. The force distribution structures may be axially distributed over the lengths of the first and second base panels.

### INCORPORATION BY REFERENCE

All publications, patents, and patent applications mentioned in this specification are herein incorporated by reference to the same extent as if each individual publication, patent, or patent application was specifically and individually indicated to be incorporated by reference.

### BRIEF DESCRIPTION OF THE DRAWINGS

The novel features of the invention are set forth with particularity in the appended claims. A better understanding of the features and advantages of the present disclosure will be obtained by reference to the following detailed description that sets forth illustrative embodiments, in which the principles of the present disclosure are utilized, and the accompanying drawings of which:

FIG. 1A shows a perspective view of a scar inhibition device, according to embodiments of the present disclosure.

FIG. 1B shows a side view of the scar inhibition device of FIG. 1A, according to embodiments of the present disclosure.

FIGS. 2A, 2B, 2C, 2D, 2E, 2F, and 2G show an exemplary method of inhibiting scar formation during healing of the closed incision, according to embodiments of the present disclosure.

FIG. 3 shows a perspective view of another scar inhibition device, according to embodiments of the present disclosure.

FIGS. 4A, 4B, 4C, and 4D show a method of applying the scar inhibition device of FIG. 3, according to embodiments of the present disclosure.

### DETAILED DESCRIPTION

The apparatus and methods of the present disclosure can be used during both the formation and the closure of surgical incisions made to a patient's skin or other tissue during surgical procedures or wounds in general. As described hereinafter, the direction of the incision or wound will define both “axial” and “lateral” directions as those terms are used herein. Most incisions will be made along a generally straight line which will define the axial direction. The lateral direction will generally be across the axial direction, typically but not necessarily being perpendicular or normal to the axial direction. Most incisions will be generally linear but in some cases the incisions could be curved or have other geometries. The term “axial” will then apply to the direction of the incision at any particular location, resulting in lateral directions which could also vary.

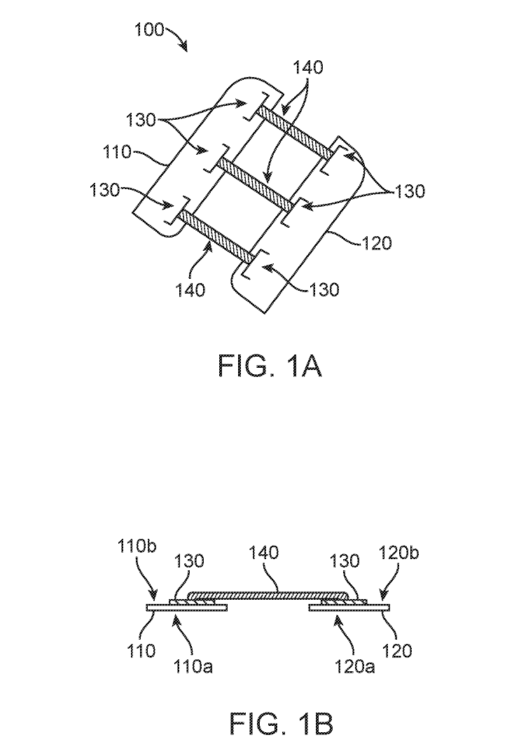

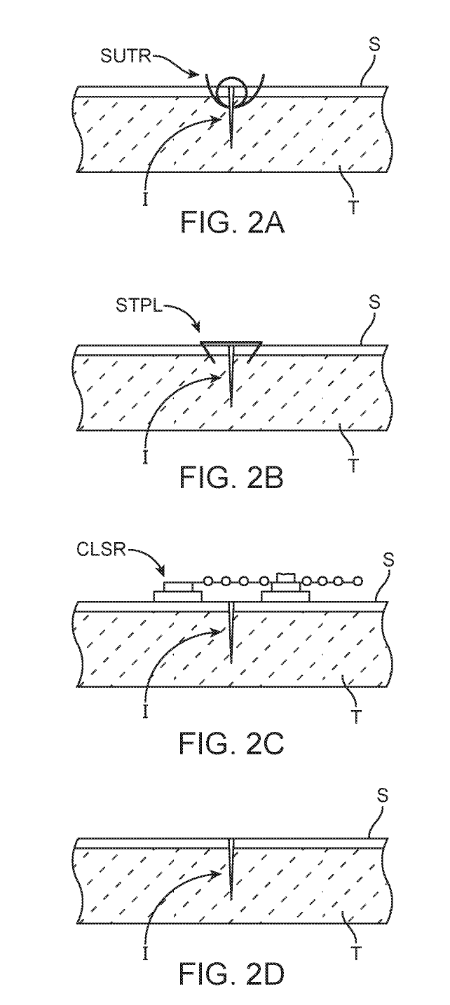

Referring now to FIGS. 1A and 1B, a scar inhibition device 100 according to embodiments of the present disclosure is shown. The scar inhibition device 100 may comprise a left base panel 110 and a right base panel 120. The left base panel 110 may comprise an adherent lower layer 110a and an upper layer 110b. Similarly, the right base panel 120 may comprise an adherent lower layer 120a and an upper layer 120b. The upper layers 110a, 120a will typically be flexible but stiff enough to be secured to tissue and minimize disruption of the healing incision or wound and surrounding tissue. The upper layers 110b, 120b may comprise a plastic layer made of rubber, latex, polyurethane, silicone, a thermoplastic elastomer, a woven fabric, a spun fabric, or similar materials. The adhesive bottom layers 110a, 120a will typically be flexible and more elastic than the upper layers 110b, 120b to follow any movement of the underlying skin and tissue to maintain adhesion, minimize blistering, and otherwise reduce irritation. The adherent lower layers 110a, 120a may comprise an adhesive material suitable for skin contact such as a hydrocolloid, a hydrogel, an acrylic polymer, silicone, poly (ethylene glycol), and the like.

The right and left base panels 110, 120 may be coupled together by a plurality of axially-distributed, elastic lateral ties 140. When the right and left base panels 110, 120 are stretched apart relative to one another and adhered to skin, the elastic lateral ties 140 may be placed in tension such that they exert a lateral compressive force on the underlying tissue. The right and left base panels 110, 120 may each comprise force distribution structures 130 which may evenly distribute the lateral compressive force exerted on the incision. Each lateral tie 130 may comprise a first end coupled to a force distribution structure 130 on the left base panel 110 and a second end coupled to an axially aligned force distribution structure 130 on the right base panel 120. Rather than being attached at two points at the base panels 110, 120, which would concentrate the forces at the two points and thereby presenting a risk of ischemia, the ends of the lateral ties 140 couple to the base panels 110, 120 through the wider area force distribution structures 130 to evenly distribute the attachment and tension forces across a larger area, thereby minimizing local stress and reducing the risk of adhesion loss and ischemia. The elastic lateral ties 140 may be made of an elastic polymer such as rubber, for example.

The force distribution structures 130 may be disposed on the base panels 110, 120 along their respective axial lengths. The force distribution structures 130 may further facilitate and limit axial and/or lateral stretching of the base panels 110, 120. For instance, the force distribution structures 130 may be made of an inelastic material that limits the stretchability of the underlying portions of the base panel 110 or 120, thereby allowing the portions of the base panel 110 or 120 uncovered by force distribution structures 130 to preferentially stretch. The force distribution structures 130 may be made of a flexible, resilient plastic, typically a stiffer plastic, such as Nylon, Polypropylene, Polyethylene, Poly carbonate, and other thermoplastic polymers. The force distribution structures 130 may have any number of shapes such as a rectangle, square, circle, triangle, a C-shape, a D-shape, or a cross, to name a few. In many embodiments, the spacing between ties 140 is 10 mm, the material of the ties is nylon, and the dimension is a round cross-section of 0.030 inch. Similar force distribution structures are described in U.S. patent application Ser. No. 14/180,564, filed 14 Feb. 2014 and issued as U.S. Pat. No. 9,089,328 on 28 Jul. 2015 [Attorney Docket No. 35383-709.502]

The base panels 110, 120 may preferentially stretch in the axial or longitudinal direction in the areas of the base panels 110, 120 between the force distribution structures 130. In some embodiments, one or more perforations may be provided in-between axially adjacent force distribution structures 130 to further facilitate the axial and/or lateral stretching of the base panels 110, 120. The perforations may be all the way through the upper layers 110b, 120b and lower layers 110a, 110b to provide aeration to the underlying tissue or may only be present on the upper layers 110b, 120b. There may be a plurality of perforations in a lateral line between the force distribution structures 130, for example. The perforations may also reduce the stress incurred as the skin stretches radially outward from the incision such as during joint articulation and swelling. Similar preferentially stretching regions and perforations are described in U.S. application Ser. No. 14/180,564, filed 14 Feb. 2014 and issued as U.S. Pat. No. 9,089,328 on 28 Jul. 2015 [Attorney Docket No. 35383-709.502].

The scar inhibition device 100 may be placed over a closed wound or incision in the skin of a patient or subject, such as skin at the patient or subject's joint, for example, the knee. In incisions placed in proximity to articulating joints, the knee in particular, device integrity is often challenged by a number of factors. These factors include longitudinal elongation, circumferential swelling, or opening of the wound as articulation occurs, skin damage such as blistering, adhesion loss, and passage of wound exudates. Joints such as knee, elbow, ankle, and shoulder may undergo a movement which can sometimes result in articulation covering more than 135° movements, leading to the challenges noted above.

In a bent position, the skin around the knee can stretch up to 50% axially (i.e., parallel to the incision) and laterally (i.e., transverse or perpendicular to the incision). An appliance adhered to the skin in this area may preferably be able to provide enough tension to maintain closure of the wound or the incision yet accommodate the stretch with minimal local stress. Minimizing the local stress may prevent local skin adhesion loss or damage to the skin if the adhesive loss does not occur. An important property for scar inhibition appliance disclosed herein is the ability of the tension load of the tension element or lateral ties 140 to be distributed across an area larger than that of the attachment point(s) themselves. Furthermore, the structure comprising the adhesive to which the lateral ties 140 are attached may in many cases have the ability to distribute the compliance of the structure across the region of skin stretch such that the appliance holds the incision in place while the skin moves around it.

The skin adhesive used in the scar inhibition device 100 at the adherent lower layers 110a, 120a may also need to withstand the elongation of the skin and be able to retract/recoil when the skin is returned to an un-stretched condition (e.g., in the fully extended knee position). Hydrocolloid adhesives may provide such properties and may be preferably suited for this application. Other adhesives such as acrylic may also be used to provide this property. In general, such adhesives may need to be attached to an elastic thin film, which may make up the upper layers 110b, 120b of the scar inhibition device 100, in order to hold their structure during expansion and recoil. Without such support, the adhesive may tear and separate with repeated elongation. The elastic thin films may comprise a thermoplastic elastomer such as polyurethane as well as various grades of silicone.

The skin adhesive used for each panel 110, 120 may preferably comprise a hydrocolloid adhesive, such as at their adherent lower layers 110a, 120a. Alternatively or in combination, the skin adhesive may comprise one of many acrylic formulations known in the art. Hydrocolloid adhesives may have the benefit of being very tacky and able to absorb moisture and shedding skin cells. Thus, hydrocolloid adhesives may be particularly suited for long-term wear applications (e.g., up to 21 days). In at least some instances, the hydrocolloid structure may be soft, however, and may be prone to creep under tension unless reinforced in some manner such as by covering the hydrocolloid adhesive layer with stiffer base panels or upper layers 110b, 120b or other covering structures disclosed herein.

One or more of the components of the scar inhibition appliances or devices disclosed herein, including one or more of the various base assemblies, base panels, force distribution structures, closure components, lateral ties, adhesive layers, etc., may be comprised of, be coated with, or otherwise incorporate one or more of an antifungal, antibacterial, antimicrobial, antiseptic, or medicated material. For example, such materials may be incorporated into the hydrocolloid adhesive layer, as another layer or coating between the skin and the adhesive layer (covering at least a portion of the adhesive layer), incorporated into the base assembly cover or at least its adhesive layer, etc. One or more wells, grooves, openings, pores, or similar structures may be provided on the device or apparatus components to facilitate such incorporation. In many embodiments, such materials may comprise one or more of silver, iodide, zinc, chlorine, copper, or natural materials such as tea tree oil as the active agent. Examples of such antifungal, antibacterial, antimicrobial, antiseptic, or medicated materials include, but are not limited to, the Acticoat™ family of materials available from Smith & Nephew plc of the U.K., the Acticoat™ Moisture Control family of materials available from Smith & Nephew plc of the U.K., the Contreet™ Foam family of materials available from Coloplast A/S of Denmark, the UrgoCell™ Silver family of materials available from Urgo Limited of the U.K. (a subsidiary of Laboratoires URGO of France), the Contreet™ Hydrocolloid family of materials available from Smith & Nephew plc of the U.K., the Aquacel™ Ag family of materials available from ConvaTec Inc. of Skillman, N.J., the Silvercel™ family of materials available from Kinetic Concepts, Inc. of San Antonio, Tex., Actisorb™ Silver 220 available from Kinetic Concepts, Inc. of San Antonio, Tex., the Urgotul™ SSD family of materials available from Urgo Limited of the U.K. (a subsidiary of Laboratoires URGO of France), the Inadine™ family of materials available from Kinetic Concepts, Inc. of San Antonio, Tex., the Iodoflex™ family of materials available from Smith & Nephew plc of the U.K., the Sorbsan Silver™ family of materials available from Aspen Medical Europe Ltd. of the U.K., the Polymem Silver™ family of materials available from Ferris Mfg. Corp. of Burr Ridge, Ill., the Promogram™ family of materials available from Kinetic Concepts, Inc. of San Antonio, Tex., the Promogram Prisma™ family of materials available from Kinetic Concepts, Inc. of San Antonio, Tex., and the Arglaes™ family of materials available from Medline Industries, Inc. of Mundelein, Ill.

In many embodiments, topical medicinal agents are incorporated directly into the scar inhibition appliance 100 described herein. Because the scar inhibition device 100 is typically applied in close proximity to a wound or incision in need of medicinal protection, the incorporation of such medicines directly into the closure device may be beneficial. In wounds at risk of infection, incorporation of anti-microbial agents may be beneficial, for example. Anti-microbial agents may include antibiotic medicines as well as antiseptic metal ions and associated compounds which may include silver, iodine, copper, and chlorine, or natural materials such as tea tree oil. In wounds prone to fungus, medicinal agents such as zinc may be warranted, for example. Combinations of any of these agents may also be of benefit and thus may be incorporated into scar inhibition device 100.

Topical medicinal agents may be incorporated into the scar inhibition device 100 in a way to give the device 100 the ability to wick exudate away from the wound (e.g., to direct unwanted organisms away from the wound and/or prevent skin maceration), while keeping the wound sufficiently hydrated for improved healing.

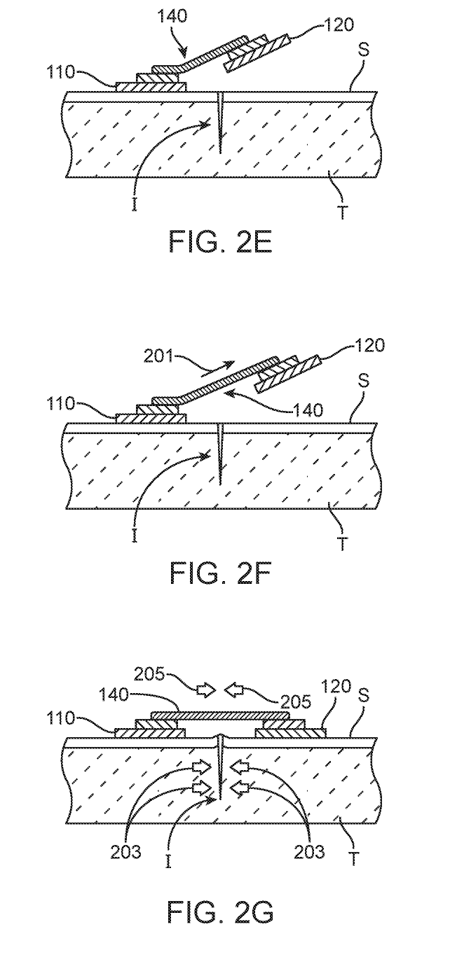

Referring now to FIGS. 2A-2G, an exemplary method of inhibiting scar formation during healing of the closed incision I is disclosed. As shown in FIGS. 2A-2D, the incision I may start out as being closed. The incision I may be closed with sutures SUTR (FIG. 2A), staples STPL (FIG. 2B), or a closure device CLSR (FIG. 2C), such as those described in U.S. Pat. Nos. 8,313,508, 8,323,313, and 8,439,945; U.S. Patent Publication No. 2013/0066365; and PCT application nos. US 2010/000430, US 2011/139912, US 2011/40213, US 2011/34649, and US 2013/067024, which are incorporated herein by reference. After the incision I has been closed for a sufficient period of time (such as 1 to 7 days), the sutures SUTR, the staples STPL, or the closure device CLSR may be removed, leaving a closed, but not fully healed incision I as in FIG. 2D. The scar inhibition device 100 may then be adhered to the skin adjacent the healing incision I. As shown in FIG. 2E, the left panel 110 may first be adhered to the skin on the left side of the incision I. As shown in FIG. 2F, the lateral ties 100 of the scar inhibition device 100 may then be placed into tension by pulling the right panel 120 away from the left panel 110 in the outward direction indicated by the arrow 201. As shown in FIG. 2G, the right panel 120 may then be adhered to the skin adjacent the healing incision I with the lateral ties 130 in tension. When tensioned, the lateral ties 140 tend to compress in inward directions indicated by arrows 205 such that the tensioned lateral ties 130 exert a laterally compressive force, indicated by arrows 203, on the tissue T adjacent the incision I. The laterally compressive force may promote healing while inhibiting scar formation. In some embodiments, the laterally compressive force may urge the skin most immediately adjacent the incision I to “pucker” upward, which may thereby inhibit scar formation as well. While the method is described above for inhibiting scar formation of an incision I, the method is equally applicable for inhibiting scar formation during the healing of any wounds on the skin in general.

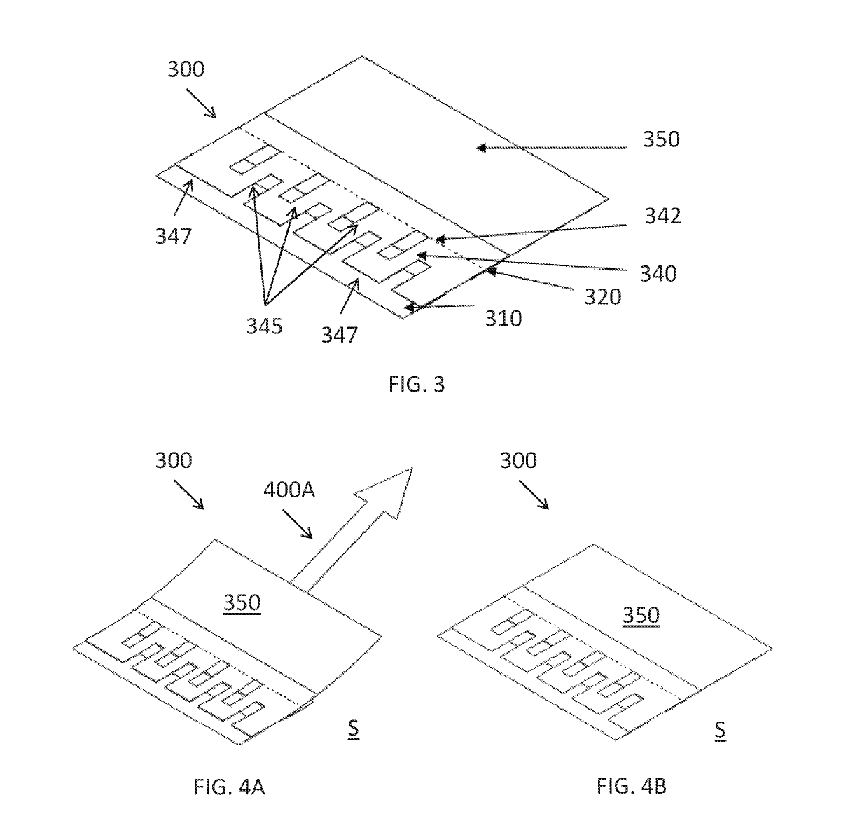

FIG. 3 shows another scar inhibition device 300 which may be used in the same way the scar inhibition device 100 is used as described above. The scar inhibition device 300 may have components similar to those of the scar inhibition device 100 described herein. The scar inhibition device 300 may comprise a left base panel 310 and a right base panel 320 to be adhered to either side of an incision I. Each of the base panels 310, 320 may comprise an adherent lower layer and an upper layer. The adherent lower layers and the upper layer may comprise any of the materials described above for the same layers in the scar inhibition device 100 (and vice versa). For instance, the adherent lower layers of the base panels 310, 320 may comprise a skin contact pressure sensitive adhesive (PSA) such as hydrocolloid, acrylic, silicone, and/or polyurethane PSAs.

Instead of a series of parallel, elastic lateral ties bringing the two base panels together, the scar inhibition device 300 comprises elastic strap component 340. As shown in FIG. 3, the elastic strap component 340 may comprise a film having lateral cut-outs thereon such that the film has a serpentine pattern, and comprises a series of lateral straps 345 and a series of side regions 347 for adhering to the upper surfaces of the base panels 310, 320 arranged in the serpentine pattern. The elastic strap component 340 may comprise an elastic film configured to apply the tension between the base panels 310, 320. The elastic strap component 340 may be made of an elastic material such as polyurethane, EVA, or silicone and may be approximately 0.75 mil to 2 mil thick.

As shown in FIG. 3, the elastic strap component 340 is adhered to or otherwise coupled to the left base panel 310. A non-elastic pull tab 350 may be coupled to the elastic strap component to tension the elastic straps. Adhesive 342 may be present on the under-side of the edge of the elastic strap component 340 and may be covered by the pull tab 350. The pull tab 350 may be pulled to apply tension to the elastic strap component 340, after which the pull tab 350 may be removed to expose the adhesive 342 which may be used to adhere the tensioned elastic strap component to the right base panel 320.

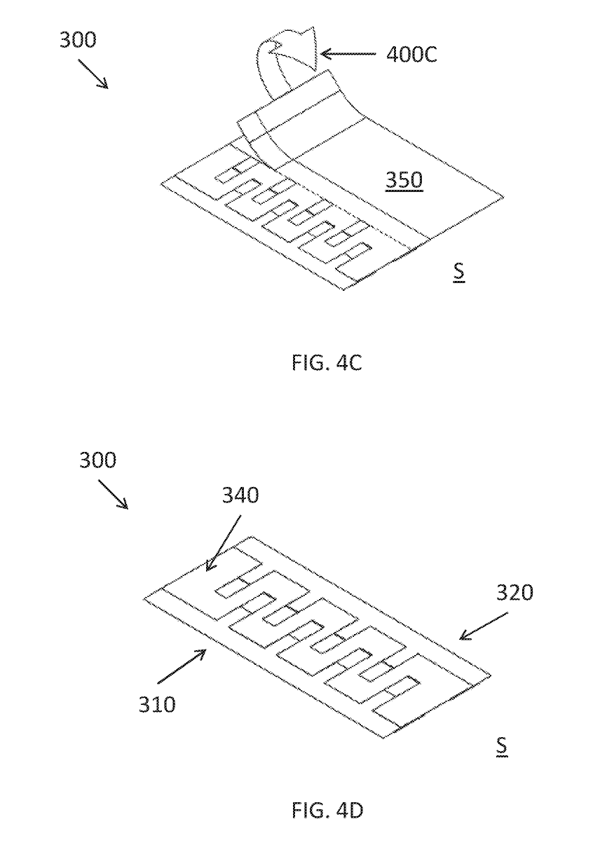

FIGS. 4A, 4B, 4C, and 4D show a method of applying the scar inhibition device 300. The device 300 may be applied to the skin S by removing a release liner from the adhesive bottom layers for the first and second base panels 310, 320, with the incision centered between the two base panels 310, 320.

Referring to FIG. 4A, a second release liner may be removed to expose the adhesive 342 under the elastic strap component 340 and the pull tab 350. The elastic strap component 340 may be tensioned by pulling the pull tab 350 perpendicularly to the incision as shown by arrow 400a.

Referring to FIG. 4B, once the desired tension is achieved, the elastic strap component 340 may be affixed to the right base panel 320 by applying the exposed adhesive 342 to the right base panel 320. The desired tension may be achieved by visual cues such as aligning the edge of the lateral straps 345 to the inner edge of the right base panel, or by aligning printed lines/dots.

Referring to FIG. 4C, the pull tab 350 may be removed from the device 300 by tearing it way (such as in a direction indicated by the arrow 400C), or tearing along perforations, or by cutting with scissors.

Referring to FIG. 4C, the scar inhibition device 300 once deployed would off-load tension from the incision.

While preferred embodiments of the present disclosure have been shown and described herein, it will be obvious to those skilled in the art that such embodiments are provided by way of example only. Numerous variations, changes, and substitutions will now occur to those skilled in the art without departing from the scope of the present disclosure. It should be understood that various alternatives to the embodiments of the present disclosure described herein may be employed in practicing the present disclosure. It is intended that the following claims define the scope of the invention and that methods and structures within the scope of these claims and their equivalents be covered thereby.

1.-14. (canceled)

15. A method of closing a wound or incision and inhibiting scar formation in the closed wound or incision, the method comprising:
adhering a first base panel of a scar inhibition device to skin on a first lateral side of the wound or incision;
stretching the first base panel and a second base panel of the scar inhibition device apart to place one or more elastic lateral ties or straps coupling the first and second base panels together in tension;
positioning the scar inhibition device such that the one or more lateral ties or straps are positioned across the wound or incision; and
adhering the second base panel to skin on a second lateral side of the wound or incision opposite the first lateral side, thereby having the one or more lateral ties apply a lateral compressive force on the wound or incision,
wherein the lateral compressive force is maintained for a period of time as the wound or incision heals.

16. The method of claim 15, wherein the period of time is in the range from 1 day to 21 days.

17. The method of claim 15, wherein the first base panel is adhered to the first lateral side of the wound or incision before the second base panel is adhered to the second lateral side of the wound or incision.

18. The method of claim 15, wherein the first and second base panels are stretched apart laterally after the first base panel is adhered to the first lateral side of the wound or incision.

19. The method of claim 15, wherein the first base panel is adhered to the first lateral side of the wound or incision concurrently with the second base panel being adhered to the second lateral side of the wound or incision.

20. The method of claim 19, wherein the first and second base panels are stretched apart before the first and second base panels are adhered to the skin.

21. The method of claim 15, wherein the one or more lateral ties comprises a plurality of axially-adjacent lateral ties or straps.

22. The method of claim 15, wherein the one or more elastic lateral ties or straps elastically apply the compressive force through the first and second base panels adhered to the skin.

23. The method of claim 15, wherein the first and second base panels each comprise force distribution structures coupling the one or more lateral ties to the first and second base panels.

24. The method of claim 23, wherein the force distribution structures are axially distributed over the lengths of the first and second base panels.

25. The method of claim 24, further comprising axially stretching one or more of the first or second base panels, the one or more second base panels having preferentially stretching regions between axially adjacent force distribution structures.

26. The method of claim 24, further comprising removing one or more of staples, sutures, or a closure device from the wound or incision or skin adjacent thereto prior to adhering the first and second base panels to the skin.

27. The method of claim 15, wherein the wound or incision is on a joint of a patient.

28. The method of claim 27, wherein the joint comprises a knee.

29. The method of claim 15, wherein applying the lateral compressive force on the wound or incision with the one or more lateral ties comprises causing the wound or incision to pucker.

30. The method of claim 15, further comprising axially stretching one or more of the first or second base panels.

31.-59. (canceled)
